(19) United States
(12) Patent Application Publication
(10) Pub. No.: US 2026/0233308 A1
(43) Pub. Date: Aug. 13, 2026

(54) Additively-Manufactured Device and Method

(71) Applicant: Orchid Orthopedic Solutions, LLC, Mason, MI (US)

(72) Inventors: Taban Larimian, Lansing, MI (US); Scott Reese, Doylestown, PA (US); Shilesh Jani, Collierville, TN (US)

(21) Appl. No.: 19/535,157

(22) Filed: Feb. 10, 2026

Related U.S. Application Data
(60) Provisional application No. 63/757,417, filed on Feb. 12, 2025.

(51) Int. Cl. B22F 10/28; B33Y 10/00; B33Y 80/00

(52) U.S. Cl. CPC B22F 10/28; B33Y 10/00; B33Y 80/00; B22F 2301/205

(57) ABSTRACT

A device includes an inner portion and an outer portion. The inner portion includes a plurality of first grains having a first size. The outer portion is disposed around the inner portion. The outer portion includes a plurality of second grains having a second size that is greater than the first size.

### CROSS-REFERENCE TO RELATED APPLICATIONS

This application claims the benefit of U.S. Provisional Application No. 63/757,417 filed Feb. 12, 2025, the entire disclosure of which is incorporated herein by reference.

### FIELD

The present disclosure relates to devices formed by additive manufacturing and, more particularly, to medical implant devices formed by hatch (e.g., line) melting and point melting.

### BACKGROUND

For products and other devices (e.g., medical implant devices), material grain characteristics play a critical role in the device's overall performance and, for example, integration with a body of a patient. While known devices and manufacturing methods for creating varying grain characteristics have proven acceptable for their intended purpose, a continuous need for improvements remains in the pertinent art to address the challenges associated with manufacturing devices with varying grain characteristics.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

One aspect of the disclosure provides a device. The device includes an inner portion and an outer portion. The inner portion includes a plurality of first grains having a first size. The outer portion is disposed around the inner portion. The outer portion includes a plurality of second grains having a second size that is greater than the first size.

Another aspect of the disclosure provides a method of manufacturing a device. The method includes forming a first layer of the device by executing a line melting process to form first grains. The first grains have a first size, and the first grains define a portion of an outer portion of the device. The method includes forming a second layer of the device by executing the line melting process to form additional first grains, and a point melting process to form second grains. The second grains have a second size that is smaller than the first size. The second grains define at least a portion of an inner portion of the device. The additional first grains define an additional portion of the outer portion. The outer portion is disposed around the inner portion.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a perspective view of an example device in accordance with the principles of the present disclosure.

FIG. 2 is a cross-sectional view of the device of FIG. 1 taken along the line 2 -2 of FIG. 1.

FIG. 3 is a cross-sectional view of the device of FIG. 1 taken along the line 3 -3 of FIG. 2.

FIGS. 4A, 4B, and 5 are schematic views illustrating example operations of manufacturing the device of FIG. 1.

FIG. 6 is a flowchart depicting an example method of manufacturing the device of FIG. 1.

In the drawings, reference numbers may be reused to identify similar and/or identical elements.

### DETAILED DESCRIPTION

The present disclosure relates to devices (e.g., medical implants), and related methods, having enhanced fatigue properties produced by a powder bed fusion (PBF) additive manufacturing (AM) method or other three-dimensional (3D) printing method. Processing parameters (e.g., themes) have a significant impact on the microstructure and grain size of 3D-printed devices. The processing parameters may include the speed (e.g., scan speed) by which a powder bed is scanned by a heat source (e.g., an electron beam), the thickness of each powder layer (e.g., layer thickness), the distance between adjacent scan paths (e.g., hatch spacing), and the pattern by which the heat source moves across the powder bed (e.g., scan strategy), among others.

In various implementations, the processing parameters dictate the amount of energy (e.g., temperature) introduced to each point (e.g., location on a powder bed) during the printing process which will in-turn alter the size of the grains of the material of the 3D-printed device. Larger amounts of energy (e.g., temperature) introduced to each point can result in enlargement of the grains and coarsening of the microstructure, whereas smaller amounts of energy typically result in a more refined microstructure and smaller grains. Size of the grains is one determining factor in the mechanical properties of metallic materials, including fatigue strength.

In fatigue loads (e.g., cyclic loads), surface cracks can initiate at sites of localized stress concentrations such as voids or grain boundaries. Therefore, a higher number of surface grain boundaries can create more opportunities for crack nucleation/initiation. Larger grains reduce the number of grain boundaries at the surface, which can increase a device's resistance to crack initiation. Crack propagation, on the other hand, has an inverse correlation with grain size. Grain boundaries act as barriers for cracks to propagate across adjacent grains, in effect acting as a crack inhibitor. For example, when a crack encounters a grain boundary, it must change direction to navigate around it, which consequently restricts the crack propagation. In a fine-grained material, there are more grain boundaries per unit volume, such that the grain boundaries act as obstacles to crack advancement.

In various implementations, materials having larger grains on an outer region and smaller grains on the inner areas resist the formation (e.g., due to less surface-emergent grain boundaries) and propagation (e.g., due to greater interior density of grain boundaries) of cracks under cyclic loads, thereby improving overall fatigue strength of the material.

The present disclosure relates to a set of processing parameters for manufacturing parts using a fusion-based AM method that generates larger grain sizes on the outer regions while having smaller grain sizes on the inside.

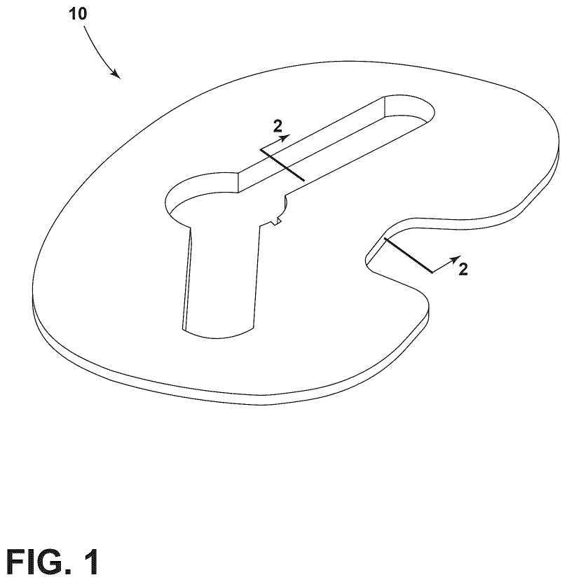

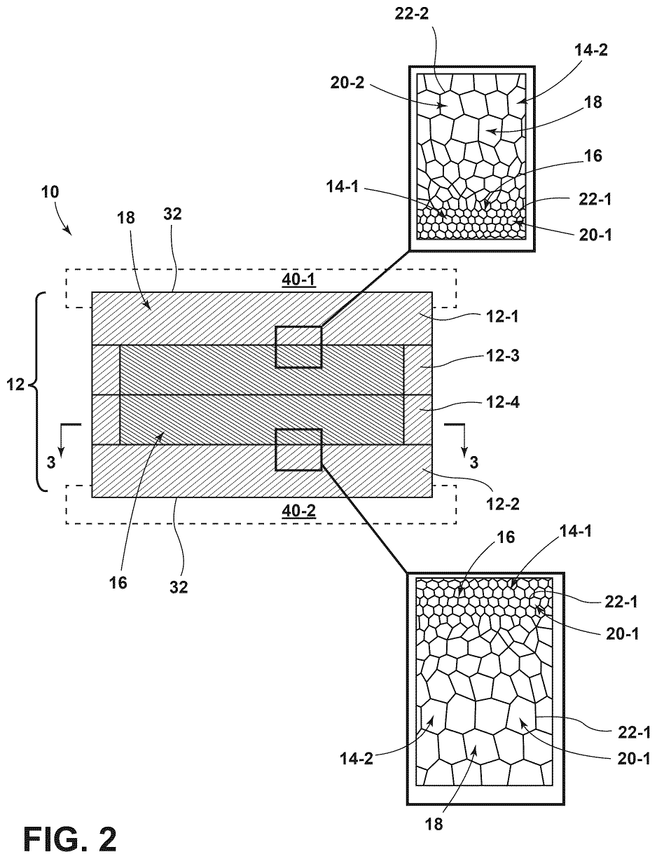

FIG. 1 illustrates an example device 10 (e.g., a medical implant). With reference to FIG. 2, in various implementations, the device 10 is defined by a set of layers 12 (collectively, layers 12). The set of layers 12 (e.g., 3D-printed layers) may include a first layer 12-1, a second layer 12-2, a third layer 12-3, and/or a fourth layer 12-4, among others.

In various implementations, the device 10 includes a plurality of first grains 14-1 and a plurality of second grains 14-1. The first grains 14-1 may define an inner portion 16 of the device 10 and the second grains 14-2 may define an outer portion 18 of the device 10. In various implementations, the outer portion 18 is disposed around the inner portion 16. For example, the outer portion 18 may enclose and/or surround the inner portion 16.

In various implementations, the first grains 14-1 have a first size 20-1. Each of the first grains 14-1 is defined by a respective first boundary 22-1. In various implementations, the first size 20-1 corresponds to a grain area enclosed by a respective first boundary 22-1. In various implementations, the first grains 14-1 inhibit crack propagation on or in the device 10. As will be explained in more detail below, the first grains 14-1 may be formed via a point melting process.

In various implementations, the second grains 14-2 have a second size 20-2 that is greater than the first size 20-1. Each of the second grains 14-2 is defined by a respective second boundary 22-2. In various implementations, the second size 20-2 corresponds to a grain area enclosed by a respective second boundary 22-2. In various implementations, the second grains 14-2 inhibit cracks from forming on the device 10. As will be explained in more detail below, the second grains 14-2 may be formed via a line melting process.

In various implementations, the first and second layers 12-2 define outer layers of the device 10. The third and fourth layers 12-3, 12-4 may define inner layers and may be disposed between the first and second layers 12-1, 12-2. The first and second layers 12-1, 12-2 may include second grains 14-2. The third and fourth layers 12-3, 12-4 may include first grains 14-1 and second grains 14-2.

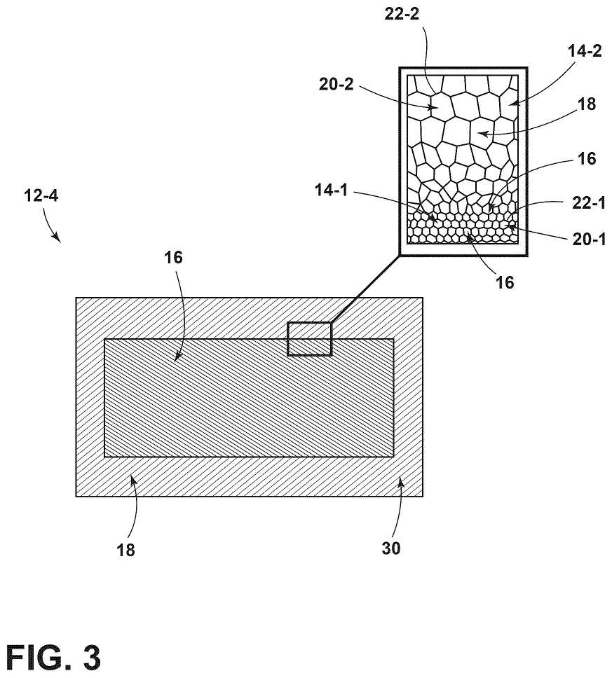

FIG. 3 illustrates an example inner layer (e.g., the fourth layer 12-4). In various implementations, the inner layer includes first grains 14-1 disposed in a central location of the layer and second grains 14-2 disposed around the first grains 14-1. The second grains 14-2 may define a peripheral portion 30 of the layer and may enclose and/or surround the first grains 14-1. While the device 10 is generally described and depicted herein as including four layers 12, the device 10 may include more or less than four layers 12.

Referring again to FIG. 2, in various implementations, at least one of the first layer 12-1 or the second layer 12-2 defines an outer surface 32. The outer surface 32 may engage at least one bone and/or another portion of a recipient of the device 10. For example, during a medical procedure (e.g., surgery), the device 10 may be coupled to, and/or replace a portion of, a first bone 40-1 or other parts (e.g., cartilage; not depicted) of a human or animal body. Upon completion of the medical procedure, the device 10 may remain coupled to the first bone 40-1 and may engage a second bone 40-2 and/or other parts (e.g., cartilage; not depicted) of the human or animal body.

While the device 10 is generally shown and described herein as being a tibia base plate implant, it will be appreciated that the device 10 may include, and/or otherwise be used in, other applications (e.g., hip, elbow, shoulder, spinal disc, etc.) within the scope of the present disclosure.

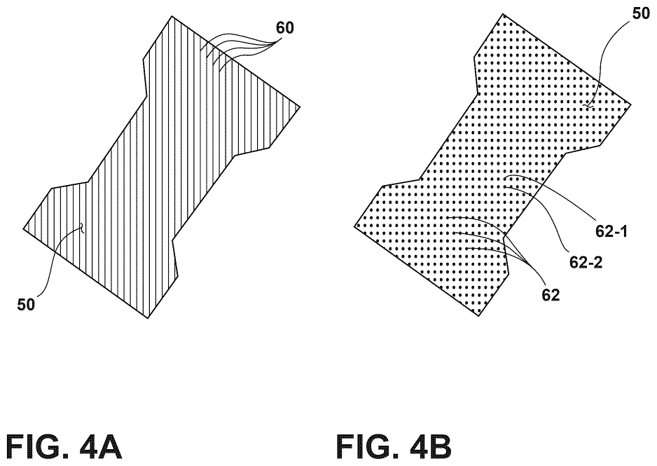

Referring now to FIG. 4A, in various implementations, the layers 12 or the portions of the layers 12 that include the second grains 14-2 may be formed via electron beam melting (EBM). For example, in EBM using a Spectra machine, the electron beam can scan a powder bed 50 by moving a beam (e.g., an electron beam) in a line 60 (e.g., linear or curvilinear) until it hatches the entire cross section (e.g., line melting process) to form the second grains 14-2. The locations and/or characteristics of the second grains 14-2 may be selectively chosen by a user.

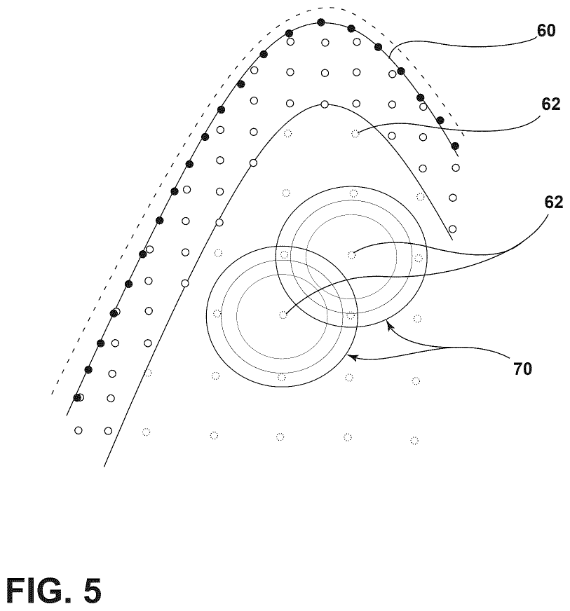

With reference to FIGS. 4B and 5, in various implementations, the layers 12 or the portions of layers 12 that include the first grains 14-1 may be formed via a second printing strategy. For example, in the second printing strategy, the electron beam can scan a powder bed 50 point-by-point until the entire intended area is covered. This second strategy is called a point melting process and forms the first grains 14-1. In the point melting process, after each point 62 (e.g., discrete location) is scanned by the beam, an inhibition zone 70 and inhibition time will come into effect which would prevent the beam from coming into a certain vicinity of the scanned point (inhibition zone) for a specific amount of time (inhibition time) (see, e.g., FIG. 5). The locations and/or characteristics of the first grains 14-1 may be selectively chosen by a user.

In various implementations, the mechanism behind the point melting process (e.g., point melt theme) prevents too much energy from being introduced to each point 62 (e.g., discrete melt location), which prevents aggressive grain growth and yields a microstructure that is finer and grains (e.g., the first grains 14-1) that are smaller than the microstructure and grains (e.g., the second grains 14-2) generated using the line melting process.

In various implementations, using the line melting process (e.g., line melt theme) for forming the outer portion 18 of the device 10 yields a coarser structure (e.g., in comparison with the point melting process) that inhibits fatigue crack initiation. On the other hand, using the point melting process with finer microstructure inhibits fatigue crack propagation. Combining the two processes to create an aggregated process (e.g., an aggregated theme) that uses the line melt parameters to form the outer portion 18 of the device 10, while using the point melt parameters to form the internal sections (e.g., inner portion 16), inhibits fatigue failures in devices that are fatigue sensitive.

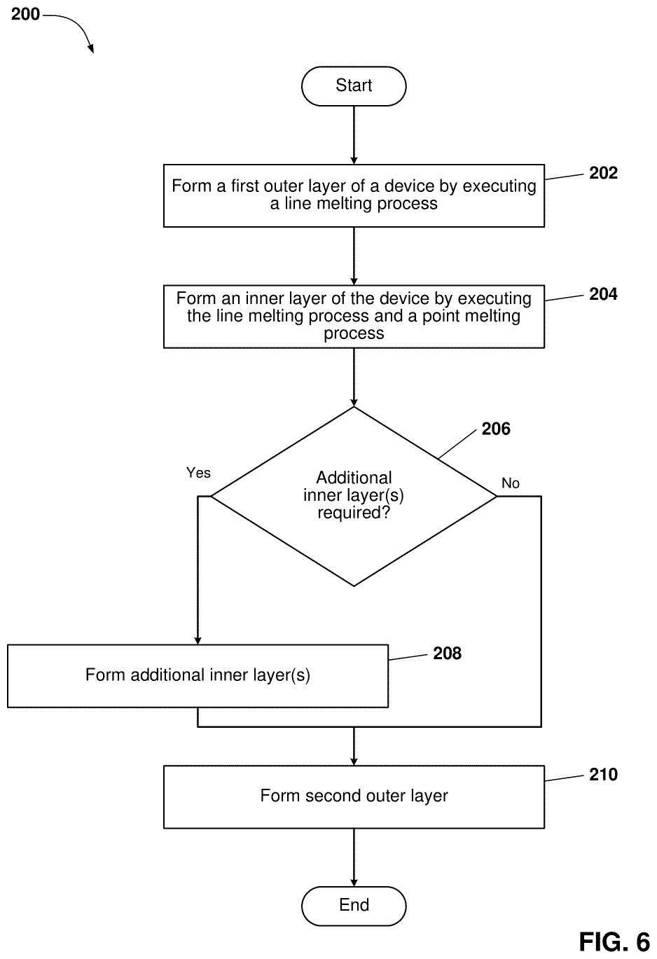

FIG. 6 is a flowchart of an example method 200 of manufacturing the device 10. The method 200 may begin at 202. At 202, a first outer layer (e.g., the first layer 12-1) is formed by executing a line melting process. The first outer layer includes second grains 14-2. In various implementations, executing the line melting process includes applying energy along a path (e.g., a line 60) in a layer of material (e.g., a powder bed 50). This process may be repeated until energy is applied along all the desired paths (see, e.g., FIG. 4A). The method 200 may proceed to 204.

At 204, an inner layer (e.g., the third layer 12-3) is formed (e.g., on top of the formed first outer layer) by executing the line melting process and a point melting process (see, e.g., FIG. 4B). The inner layer includes first grains 14-1 and second grains 14-2. In some example configurations, the second grains 14-2 of the inner layer define a peripheral portion 30 that surrounds and/or encloses the first grains 14-1 of the inner layer.

In various implementations, executing the point melting process includes applying energy to a first discrete location (e.g., a first point 62-1) in a layer of material (e.g., powder bed 50), and applying energy to a second discrete location (e.g., a second point 62-2) in the layer of material. The second location is spaced apart from the first location (see, e.g., FIG. 4B). This process may be repeated until energy is applied to all the desired discrete locations.

In various implementations, executing the line melting process in connection with forming the inner layer includes applying energy along a path (e.g., a line 60) in the layer of material. In various implementation, the path is spaced apart from the first location (e.g., the first point 62-1) and the second location (e.g., a second point 62-2). This process may be repeated until energy is applied along all the desired paths. Although the point melting process is described as being executed before the line melting process in forming the inner layer, the order of execution may be reversed. The method 200 may proceed to 206.

At 206, the method 200 includes determining whether additional inner layer(s) is/are required. For example, some devices may require multiple inner layers based on the specific application or the overall device size. This determination may be based on user input. If yes at 206, the method 200 may proceed to 208. Otherwise, the method 200 may proceed to 210.

At 208, the additional inner layers(s) 12 (e.g., the fourth layer 12-4) are formed (e.g., on the formed inner layer). For example, in response to additional inner layers(s) 12 being required, step 204 may be repeated until the desired number of additional inner layers are formed. Then, the method 200 may proceed to 210.

At 210, a second outer layer (e.g., the second layer 12-2) is formed (e.g., on the previously formed inner layer). Step 202 may be repeated to form the second outer layer. In various implementations, after printing all desired layers 12, the first grains 14-1 collectively define the inner portion 16 of the device 10, and the second grains 14-2 collectively define the outer portion 18 of the device 10. Then, the method 200 may end.

The present disclosure is not limited to electron beam melting and can be used with any powder bed technology. The processing parameters are not limited to point melt and line melt themes and can be any parameter that can generate different grain sizes. This technique can be used to generate a desired microstructure in specific regions of a device.

Titanium and its alloys, while offering excellent properties such as high strength-to-weight ratio, corrosion resistance, and heat resistance, may require coatings to improve their fatigue performance in certain applications. Titanium naturally forms a protective oxide layer (typically around 1-2 microns thick), which is generally beneficial for corrosion resistance. However, this oxide layer can be brittle under certain conditions, particularly at higher stress concentrations. Therefore, applying a layer of coating by means of plasma spray coatings, physical vapor deposition (PVD), anodizing, or shot peening, etc. will enhance the material's overall fatigue performance. However, applying a coating is an added step to the production of fatigue sensitive AM-manufactured parts, which can be both time consuming and expensive.

The method 200 described herein (e.g., a combination of line melting and point melting) eliminates the need for additional coating steps in AM-fabricated devices and improves fatigue life.

In various implementations, the point melting processes (e.g., Selective Laser Melting (SLM)) generally involves localized heating of a very small area (a “point”) by the laser or energy source prior to removing the heat (e.g., turning off the energy source) from the point. This highly localized heating results in a rapid cooling rate once the heat source moves away or is otherwise removed. Rapid cooling rates as well as existence of inhibition time and zone which prevents the heat source from quickly re-heating and coarsening the microstructure leads to finer microstructures. Therefore, for applications where more strength and slower fatigue crack propagation is required, a point melt theme may be a better choice.

Printing sections of a device using the line melting process increases the speed of production while enhancing the mechanical properties compared to devices that only use the point melting process.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

Spatial and functional relationships between elements (for example, between modules, layers, etc.) are described using various terms, including “connected,” “engaged,” “coupled,” “adjacent,” “proximate,” “next to,” “on top of,” “above,” “below,” and “disposed.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present between the first and second elements. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The term “set” does not necessarily exclude the empty set —in other words, in some circumstances a “set” may have zero elements. The term “non-empty set” may be used to indicate exclusion of the empty set —in other words, a non-empty set will always have one or more elements. The term “subset” does not necessarily require a proper subset. In other words, a “subset” of a first set may be coextensive with (equal to) the first set. Further, the term “subset” does not necessarily exclude the empty set —in some circumstances a “subset” may have zero elements.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR.

Various example embodiments of the invention are described in the following clauses.

Clause 1: A device comprising: an inner portion including a plurality of first grains having a first size; and an outer portion disposed around the inner portion and including a plurality of second grains having a second size that is greater than the first size.

Clause 2: The device of clause 1, wherein the first grains are formed via a point melting process.

Clause 3: The device of any of clauses 1 through 2, wherein the second grains are formed via a line melting process.

Clause 4: The device of any of clauses 1 through 3, wherein the first grains are configured to inhibit crack propagation on or in the device.

Clause 5: The device of any of clauses 1 through 4, wherein the second grains are configured to inhibit cracks from forming on the device.

Clause 6: The device of any of clauses 1 through 5, wherein: the device includes a first layer and a second layer, the first layer includes a portion of the second grains, and the second layer includes at least a portion of the first grains and an additional portion of the second grains.

Clause 7: The device of clause 6, wherein: the device is a medical implant, the first layer defines an outer surface, and the outer surface is configured to engage a bone of a recipient of the medical implant.

Clause 8: The device of any of clauses 6 through 7, wherein: the device includes a third layer, and the second layer is disposed between the first layer and the third layer.

Clause 9: The device of clause 8, wherein the third layer includes another portion of the second grains.

Clause 10: The device of any of clauses 6 through 9, wherein the additional portion of the second grains defines a peripheral portion of the second layer.

Clause 11: The device of clause 10, wherein the peripheral portion surrounds the first grains of the second layer.

Clause 12: The device of any of clauses 1 through 11, wherein the outer portion encloses the inner portion.

Clause 13: A method of manufacturing a device, the method comprising: forming a first layer of the device by executing a line melting process to form first grains, wherein: the first grains have a first size, and the first grains define a portion of an outer portion of the device; and forming a second layer of the device by executing: the line melting process to form additional first grains, and a point melting process to form second grains, wherein: the second grains have a second size that is smaller than the first size, the second grains define at least a portion of an inner portion of the device, the additional first grains define an additional portion of the outer portion, and the outer portion is disposed around the inner portion.

Clause 14: The method of clause 13, wherein executing the line melting process to form the first layer includes applying energy along at least one path in a layer of material.

Clause 15: The method of clause 14, wherein the layer of material includes a powder bed.

Clause 16: The method of any of clauses 13 through 15, wherein: executing the point melting process in connection with forming the second layer includes: applying energy to a first discrete location in a layer of material, and applying energy to a second discrete location in the layer of material, and the second discrete location is spaced apart from the first discrete location.

Clause 17: The method of clause 16, wherein: executing the line melting process in connection with forming the second layer includes applying energy along a path in the layer of material, and the path is spaced apart from the first location and the second location.

Clause 18: The method of any of clauses 13 through 17, wherein the first grains are configured to inhibit cracks from forming on the device.

Clause 19: The method of any of clauses 13 through 18, wherein the second grains are configured to inhibit crack propagation on or in the device.

Clause 20: The method of any of clauses 13 through 18, further comprising forming a third layer of the device by executing the line melting process, the second layer is disposed between the first layer and the third layer.

1. A device comprising:
an inner portion including a plurality of first grains having a first size; and
an outer portion disposed around the inner portion and including a plurality of second grains having a second size that is greater than the first size.

2. The device of claim 1 wherein the first grains are formed via a point melting process.

3. The device of claim 1 wherein the second grains are formed via a line melting process.

4. The device of claim 1 wherein the first grains are configured to inhibit crack propagation on or in the device.

5. The device of claim 1 wherein the second grains are configured to inhibit cracks from forming on the device.

6. The device of claim 1 wherein:
the device includes a first layer and a second layer,
the first layer includes a portion of the second grains, and
the second layer includes at least a portion of the first grains and an additional portion of the second grains.

7. The device of claim 6 wherein:
the device is a medical implant,
the first layer defines an outer surface, and
the outer surface is configured to engage a bone of a recipient of the medical implant.

8. The device of claim 6 wherein:
the device includes a third layer, and
the second layer is disposed between the first layer and the third layer.

9. The device of claim 8 wherein the third layer includes another portion of the second grains.

10. The device of claim 6 wherein the additional portion of the second grains defines a peripheral portion of the second layer.

11. The device of claim 10 wherein the peripheral portion surrounds the first grains of the second layer.

12. The device of claim 1 wherein the outer portion encloses the inner portion.

13. A method of manufacturing a device, the method comprising:
forming a first layer of the device by executing a line melting process to form first grains, wherein:
the first grains have a first size, and
the first grains define a portion of an outer portion of the device; and
forming a second layer of the device by executing:
the line melting process to form additional first grains, and
a point melting process to form second grains, wherein:
the second grains have a second size that is smaller than the first size,
the second grains define at least a portion of an inner portion of the device,
the additional first grains define an additional portion of the outer portion, and
the outer portion is disposed around the inner portion.

14. The method of claim 13 wherein executing the line melting process to form the first layer includes applying energy along at least one path in a layer of material.

15. The method of claim 14 wherein the layer of material includes a powder bed.

16. The method of claim 13 wherein:
executing the point melting process in connection with forming the second layer includes:
applying energy to a first discrete location in a layer of material, and
applying energy to a second discrete location in the layer of material, and
the second discrete location is spaced apart from the first discrete location.

17. The method of claim 16 wherein:
executing the line melting process in connection with forming the second layer includes applying energy along a path in the layer of material, and
the path is spaced apart from the first location and the second location.

18. The method of claim 13 wherein the first grains are configured to inhibit cracks from forming on the device.

19. The method of claim 13 wherein the second grains are configured to inhibit crack propagation on or in the device.

20. The method of claim 13 further comprising forming a third layer of the device by executing the line melting process,
the second layer is disposed between the first layer and the third layer.
